(19) United States
(12) Patent Application Publication
(10) Pub. No.: US 2011/0189682 A1
(43) Pub. Date: Aug. 4, 2011

(54) TOOLS FOR OBJECTIVELY DETERMINING SEVERITY OF SYSTEMIC SCLEROSIS

(76) Inventor: Robert A. Lafyatis, Arlington, MA (US)

(21) Appl. No.: 13/011,814

(22) Filed: Jan. 21, 2011

Related U.S. Application Data
(60) Provisional application No. 61/336,361, filed on Jan. 21, 2010.

(51) Int. Cl. C12Q 1/68

(52) U.S. Cl. 435/6.12

(57) ABSTRACT

Methods are disclosed for the identification and use of biomarkers in order to objectively measure systemic sclerosis. The biomarker may be a single gene, or may be a combination of multiple genes. A preferred embodiment in which the biomarker is a four gene combination of two TGFβ regulated genes (COMP and THS1), and two IFN regulated genes (IFI44 and SIG1) is the best identified predictor of systemic sclerosis as measured by the modified Rodnan skin score.

### RELATED APPLICATION DATA

This application claims the benefit under 35 U.S.C. §119(e) of U.S. Provisional Application No. 61/336,361, filed Jan. 21, 2010, the content of which is hereby expressly incorporated herein by reference in its entirety.

### FIELD OF THE DISCLOSURE

Aspects of this disclosure relate to methods for determining the severity of systemic sclerosis. Other aspects relate to methods and tools for providing objective severity data and using the data for drug discovery trials, diagnostics, and therapeutics.

### DESCRIPTION OF BACKGROUND INFORMATION

Systemic sclerosis can be a debilitating disease. People suffering from systemic sclerosis are afflicted with fibrosis, degenerative changes, and vascular abnormalities in their skin, joints and various internal organs such as the esophagus, lower GI tract, lungs, heart, and kidneys. They often suffer from Raynaud's syndrome, polyarthralgia, dysphagia, and heartburn. Swelling and eventually skin tightening and contractures of the fingers are other common symptoms. Systemic sclerosis can even be fatal, with lung, heart, and kidney troubles accounting for most deaths.

Pulmonary arterial hypertension (PAH) is an additional common complication of systemic sclerosis. PAH is associated with high mortality rates despite modest improvements in survival due to increased screening and treatment. PAH occurs more frequently in limited systemic scleroderma patients than in those suffering from diffuse systemic scleroderma. [Steen V, Medsger T A, Jr. (2003) Predictors of isolated pulmonary hypertension in patients with systemic sclerosis and limited cutaneous involvement. Arthritis Rheum 48: 516-522].

Currently there is no effective treatment for systemic sclerosis, a disease which is associated with considerable morbidity and mortality. One can therefore understand why, although systemic sclerosis is a relatively uncommon disease (˜1:5,000), there is great interest in developing therapeutics to treat those afflicted. This interest has also grown rapidly over the last several years as the market for therapeutics targeting more common rheumatic diseases has become crowded. As a result, both humanitarian and economic motivations exist which are causing the disease to draw more attention.

### SUMMARY OF THE DISCLOSURE

At present, due to the lethality and rarity of systemic sclerosis, a large percentage of the care for patients with the disease is handled in specialized centers. The development of tools to objectively measure the degree of the disease—and of the change of the degree of the disease over time and/or in response to medication—would provide a common basis for measurement of the disease stage in doctors' offices across the country or the world. This, in turn, would allow for effective treatment outside of such specialized centers, as well as improved testing of therapeutics.

The present disclosure relates to methods for identification and use of biomarkers as objective indicators of the severity of systemic sclerosis. The disclosure also provides methods for identifying and measuring such biomarkers in one or more biopsies or human fluids for the purpose of diagnosing this disease and evaluating disease response to drugs or other therapies. These methods are useful, among other ways, as outcome measures for clinical trials of therapeutics, and also by physicians testing patients. Per one example embodiment herein, a method is provided for identifying biomarkers for systemic sclerosis. The method comprises several steps. The first step is evaluating subjects for systemic sclerosis using the modified Rodnan Skin Score (the mRSS). The next steps are taking samples from those subjects, and measuring the levels of expression of genes in the sample. The following step is to normalize the levels of expression of genes assayed in the samples, and to calculate changes in the relative expression of each gene. The next step is determining the statistical significance for differences between systemic sclerosis and control gene expression. The next step is evaluating the correlation between expression of each gene found increased in systemic sclerosis skin with the clinical outcome, the mRSS. The next step is developing a predictive model of the mRSS based on multiple linear regression analyses using the level of expression of multiple genes shown individually to correlate with the mRSS, and finally, validating the predictor.

### BRIEF DESCRIPTION OF THE DRAWINGS

FIG. 1 is a table depicting correlation levels between the mRSS and gene expression in the skin.

FIGS. 2A-2D depicts linear regression of mRNA expression of various genes in either lesional or non-lesional skin related to the mRSS, in subjects with diffuse cutaneous systemic sclerosis.

FIGS. 3A-3D depict levels of change in expression of IFN-regulated genes in skin from patients with diffuse cutaneous systemic sclerosis, in relation to the mRSS for those same subjects. Linear regression may be performed on the expression values shown in FIGS. 3A-3D to analyze the relationship between the actual expression values and the corresponding mRSS values, using a simple linear model—the model being depicted in each of the graphs in FIGS. 3A-3D as a straight line.

FIGS. 4A and 4B depict multiple linear regression of a four-gene biomarker with the mRSS in patients with diffuse cutaneous systemic sclerosis. FIG. 4A depicts a best-fit model of skin gene expression of COMP, THS1, IFI44 and SIG1 with the mRSS. FIG. 4B depicts the contribution of expression of each gene, COMP, THS1, IFI44 and 24 SIG1, to the biomarker predicted skin score. Each bar represents the biomarker predicted skin score of one patient.

FIGS. 5A and 5B depict data produced using one embodiment of a process for validating the use of the illustrated four-gene biomarker for predicting an mRSS skin score. FIG. 5A depicts a 4-gene biomarker prediction of the skin score using skin biopsies (n=12), independent from those used to develop the biomarker. The biomarker skin score was calculated from normalized mRNA expression of COMP, THS1, IFI44 and SIG1, using one embodiment of a best-fit equation determined by multiple linear regression shown in FIGS. 3A-3D. FIG. 5B depicts comparison of changes in the mRSS score values with changes in the four-gene biomarker (BSS) score values over time in 5 patients with diffuse cutaneous systemic sclerosis. Patients A and B, and C, D and E show biomarker results and the mRSS results, respectively, at baseline, 6 and 12 months, or at baseline and 6 months.

### DETAILED DESCRIPTION

The modified Rodnan skin score (the mRSS) is currently the standard primary outcome measure in most recent studies of therapeutics targeting skin disease for diffuse cutaneous systemic sclerosis. However, the mRSS is problematic due to inter and intra observer variability. [Clements P, Lachenbruch P, Siebold J, White B, Weiner S, et al. (1995) Inter and intraobserver variability of total skin thickness score (modified Rodnan TSS) in systemic sclerosis. J Rheumatol 22: 1281-1285]. Consistent evaluation of the mRSS requires careful training of scorers, and even with such training, evaluation is still prone to variability. [Czirjak L, Nagy Z, Aringer M, Riemekasten G, Matucci-Cerinic M, et al. (2007) The EUSTAR model for teaching and implementing the modified Rodnan skin score in systemic sclerosis. Ann Rheum Dis 66: 966-969]. Inter-observer scoring variability is even higher than intra-observer variability. [Pope J E, Baron M, Bellamy N, Campbell J, Carette S, et al. (1995) Variability of skin scores and clinical measurements in scleroderma. J Rheumatol 22: 1271-1276]. Studies that use the mRSS therefore require consistent scorers at clinical study sites, adding variability to scoring in studies across multiple centers.

These limitations of the mRSS contribute to the difficulty in showing efficacy of therapeutics in systemic sclerosis trials. The mRSS is not commonly employed in the clinical setting in part because of these limitations. For these reasons, among others, improving outcome measures in systemic sclerosis will likely further efforts to discover active therapeutics for the disease.

Biomarkers may, at times, be useful as supplements to clinical evaluations. Objective biomarkers may be particularly useful in connection with rheumatologic diseases, in which clinical disease may be hard to define accurately and quantitatively. Such measures can be especially useful in clinical trials, in which changes in disease status must be assessed over time. Outcome measures with increased accuracy and precision provide better discrimination of change, resulting in the potential to decrease subject enrollment an improve study efficiency. The difficulty in carrying out large studies on systemic sclerosis patients makes better outcome measure particularly important.

The amount of myofibroblasts found in the skin and expression of one TGFβ-regulated gene, cartilage oligomeric protein (COMP), correlate highly with the mRSS. [Kissin E Y, Merkel P A, Lafyatis R (2006) Myofibroblasts and hyalinized collagen as markers of skin disease in systemic sclerosis. Arthritis Rheum 54: 3655-3660; 18. Farina G, Lemaire R, Pancari P, Bayle J, Widom R L, et al. (2008) Cartilage-oligomericmatrix protein expression in systemic sclerosis reveals heterogeneity of dermal fibroblast responses to transforming growth factor-{beta}. Ann Rheum Dis.]

A number of genes have been shown to have some level of correlation with the mRSS. Some patients with systemic sclerosis show increased expression of IFN-regulated genes in peripheral blood mononuclear cells (PBMCs). [York M R, Nagai T, Mangini A J, Lemaire R, van Seventer J M, et al. (2007) A macrophage marker, Siglec-1, is increased on circulating monocytes in patients with systemic sclerosis and induced by type I interferons and toll-like receptor agonists. Arthritis Rheum 56: 1010-1020].

A variety of changes in cytokines and collagen metabolites have been found in sera and/or urine from patients with systemic sclerosis. Systemic sclerosis patients have on average increased levels of endothelin-1, interleukins-4, -6, -10, -12, -13, -17, and MCP-1. [Scala E, Pallotta S, Frezzolini A, Abeni D, Barbieri C, et al. (2004) Cytokine and chemokine levels in systemic sclerosis: relationship with cutaneous and internal organ involvement. Clin Exp Immunol 138: 540-546; Sato S, Hasegawa M, Takehara K (2001) Serum levels of interleukin-6 and interleukin-10 correlate with total skin thickness score in patients with systemic sclerosis. J Dermatol Sci 27: 140-146; Vancheeswaran R, Magoulas T, Efrat G, Wheeler-Jones C, Olsen I, et al. (1994) Circulating endothelin-1 levels in systemic sclerosis subsets—a marker of fibrosis or vascular dysfunction? J Rheumatol 21: 1838-1844].

Collagen metabolites, serum amino-terminal procollagens type I and III may also correlate with the mRSS at some level. [Lee Y J, Shin K C, Kang S W, Lee E B, Kim H A, et al. (2001) Type III procollagen N-terminal propeptide, soluble interleukin-2 receptor, and von Willebrand factor in systemic sclerosis. Clin Exp Rheumatol 19: 69-74; Scheja A, Wildt M, Wollheim F A, Akesson A, Saxne T (2000) Circulating collagen metabolites in systemic sclerosis. Differences between limited and diffuse form and relationship with pulmonary involvement. Rheumatology (Oxford) 39: 1110-1113].

Also, urinary desmosine and isodesmosine are increased in systemic sclerosis patients. [Stone P J, Korn J H, North H, Lally E V, Miller L C, et al. (1995) Cross-linked elastin and collagen degradation products in the urine of patients with scleroderma. Arthritis Rheum 38: 517-524]. These markers show a limited correlation with the mRSS. [Scheja A, Wildt M, Wollheim F A, Akesson A, Saxne T (2000) Circulating collagen metabolites in systemic sclerosis. Differences between limited and diffuse form and relationship with pulmonary involvement. Rheumatology (Oxford) 39: 1110-1113; Denton C P, Merkel P A, Furst D E, Khanna D, Emery P, et al. (2007) Recombinant human anti-transforming growth factor betal antibody therapy in systemic sclerosis: a multicenter, randomized, placebo-controlled phase I/II trial of CAT-192. Arthritis Rheum 56: 323-333].

While these markers have been shown to have some correlation to the mRSS, the extent of that correlation has been limited, and they have not heretofore been generally embraced for use in systemic sclerosis.

Other methods to assess skin involvement have been examined, particularly durometry, which shows a high interobserver intraclass correlation coefficient. [Kissin E Y, Schiller A M, Gelbard R B, Anderson J J, Falanga V, et al. (2006) Durometry for the assessment of skin disease in systemic sclerosis. Arthritis Rheum 55: 603-609; Merkel P A, Silliman N P, Denton C P, Furst D E, Khanna D, et al. (2008) Validity, reliability, and feasibility of durometer measurements of scleroderma skin disease in a multicenter treatment trial. Arthritis Rheum 59: 699-705]. Durometry also shows a moderate correlation with the mRSS.

In accordance with one embodiment, candidate sets of systemic sclerosis outcome measure biomarkers may be identified using a process. In that process, RNA is isolated from skin samples biopsied from a single location on the forearm, namely the dorsal mid-forearm, of a group of patients with diffuse cutaneous systemic sclerosis. These samples are studied for expression levels of a set of genes, specifically (per one embodiment) transforming growth factor-beta (TGFβ) and interferon (IFN)-regulated genes. The levels of select genes from among the expressed genes (per one example embodiment, one or both sets of TGFβ and IFN-regulated genes) are compared with the mRSS (e.g., using linear regression analysis) to assess the extent of correlation to corresponding values of the mRSS. Those with stronger correlations, e.g., TGFβ-regulated genes CTGF, THS1, COL4 and PAI1 and IFN-regulated genes, IFI44, SIG1, OAS2, and MX1, are further analyzed (e.g., using linear or multiple regression analysis) for predicting and/or supplementing (including, where appropriate, replacing) the mRSS. (Per one example embodiment, described further below in example 4, skin expression of TGFβ-regulated genes, cartilage oligomeric matrix protein (COMP) and thrombospondin-1 (THS1 or TSP-1) correlated moderately well with the mRSS. IFN-regulated genes were also found to correlate with the mRSS and the addition of interferon-induced 44 (IFI44) and sialoadhesin (SIG1) to COMP and THS1 in multiple regression analyses significantly improved best-fit models achieving an R²=0.89.)

From these results, a number of individual gene biomarkers correlating with the severity of system sclerosis and models for producing system sclerosis score data (e.g., equations for producing score values as a function of expression levels of the identified biomarker genes) were identified. Combinations of those biomarkers which also correlated with the mRSS are also identified. The following examples illustrate various gene sets and models for producing systemic sclerosis severity values useful for predicting and/or supplementing the mRSS.

### Example 1

In the first example, any one identified gene can be useful for predicting and/or supplementing the mRSS. The correlation between each gene and the mRSS is expressed in the listed R² values of the table:

| Gene | One Gene (R²) |
|---|---|
| COMP | 0.64 |
| THS1 | 0.32 |
| SIG1 | 0.17 |
| IFI44 | 0.41 |
| MX1 | 0.30 |
| OAS2 | 0.33 |

### Example 2

In the second example, pair combinations of each gene can be useful for predicting and/or supplementing the mRSS. The correlation between each gene pair and the mRSS is expressed in the listed R² values of the table. Each gene listed in column A is paired with the genes listed in columns B, C, D, E, and F, and the R² values for the respective combinations are listed where the row for each gene listed in column A intersects the column below each gene in the columns B-F. The R² value for each combination is listed only once.

| Gene | Two Gene Fits (R²) | | | | |
|---|---|---|---|---|---|
| A | B | C | D | E | F |
| COMP | COMP | THS1 | SIG1 | IFI44 | MX1 |
| THS1 | 0.71 | | | | |
| SIG1 | 0.66 | 0.47 | | | |
| IFI44 | 0.71 | 0.76 | 0.53 | | |
| MX1 | 0.64 | 0.61 | 0.37 | 0.43 | |
| OAS2 | 0.67 | 0.55 | 0.38 | 0.48 | 0.35 |

### Example 3

In the third example, three-gene combinations can be useful for predicting and/or supplementing the mRSS. The correlation between each three gene combination and the mRSS is expressed in the listed R² values of the table. Each gene listed in column A is combined with the genes pairs listed in columns B-K, and the R² values for the respective combinations are listed where the row for each gene listed in column A intersects the column below each gene pair in the columns B-K. The R² value for each combination is listed only once.

| | Three Gene Fits (R²) | | | | | | | | | |
|---|---|---|---|---|---|---|---|---|---|---|
| | B | C | D | E | F | G | H | I | J | K |
| Gene | COMP/ | COMP/ | COMP/ | COMP/ | THS1/ | THS1/ | THS1/ | SIG1/ | SIG1/ | IFI44/ |
| A | THS1 | SIG1 | IFI44 | MX1 | SIG1 | IFI44 | MX1 | IFI44 | MX1 | MX1 |
| SIG1 | 0.74 | | | | | | | | | |
| IFI44 | 0.84 | 0.73 | | | 0.86 | | | | | |
| MX1 | 0.73 | 0.66 | 0.75 | | 0.71 | 0.78 | | 0.53 | | |
| OAS2 | 0.69 | 0.68 | 0.72 | 0.70 | 0.61 | 0.77 | 0.65 | 0.54 | 0.40 | 0.48 |

The p-values for the three gene combinations with the highest R² values are as follows:

| | p-value | | p-value | | p-value | | p-value | R² |
|---|---|---|---|---|---|---|---|---|
| COMP: | 0.012 | THS1: | 0.003 | | | IFI44: | 0.003 | 0.84 |
| | | THS1: | <0.001 | SIG1: | <0.001 | IFI44: | 0.004 | 0.86 |

### Example 4

In the fourth example, 4-gene combinations can be useful for predicting and/or supplementing the mRSS. The correlation between each four gene combination and the mRSS is expressed in the listed R² values of the table. Each gene listed in column A is combined with the three gene set listed in columns B-J, and the R² values for the respective combinations are listed where the row for each gene listed in column A intersects the column below each three gene set in the columns B-J. The R² value for each combination is listed only once.

| | Four Gene Fits (R²) | | | | | | | | |
|---|---|---|---|---|---|---|---|---|---|
| | B | C | D | E | F | G | H | I | J |
| | COMP/ | COMP/ | COMP/ | COMP/ | COMP/ | COMP/ | THS1/ | THS1/ | SIG1/ |
| Gene | THS1/ | THS1/ | THS1/ | SIG1/ | SIG1/ | IFI44/ | SIG1/ | IFI44/ | IFI44/ |
| A | SIG1 | IFI44 | MX1 | IFI44 | MX1 | MX1 | IFI44 | MX1 | MX1 |
| IFI44 | 0.89 | | | | | | | | |
| MX1 | 0.76 | 0.85 | | 0.78 | | | 0.86 | | |
| OAS2 | 0.76 | 0.84 | 0.74 | 0.73 | 0.71 | 0.81 | 0.86 | 0.78 | 0.55 |

The p-values for the four gene combinations with the highest R² values are as follows:

| | p- | | p- | | p- | | p- | | p- | | p- | |
|---|---|---|---|---|---|---|---|---|---|---|---|---|
| | value | | value | | value | | value | | value | | value | R² |
| COMP: | 0.062 | THS1: | <0.001 | SIG1: | 0.021 | IFI44: | 0.001 | | | | | 0.89 |
| COMP: | 0.038 | THS1: | 0.015 | | | IFI44: | 0.008 | MX1: | 0.667 | | | 0.85 |
| COMP: | 0.026 | THS1: | 0.006 | | | IFI44: | 0.012 | | | OAS2: | 0.742 | 0.84 |
| COMP: | <0.001 | | | | | IFI44: | 0.018 | MX1: | 0.024 | OAS2: | 0.065 | 0.81 |
| | | THS1: | <0.001 | SIG1: | 0.013 | IFI44: | <0.001 | | | OAS2: | 0.880 | 0.86 |
| | | THS1: | <0.001 | SIG1: | 0.017 | IFI44: | 0.002 | MX1: | 0.598 | | | 0.86 |

### Example 5

In the fifth example, 5-gene combinations can be useful for predicting and/or supplementing the mRSS. The correlation between each five gene combination and the mRSS is expressed in the listed R² values of the table. Each gene listed in column A is combined with the four gene set listed in columns B-F, and the R² values for the respective combinations are listed where the row for each gene listed in column A intersects the column below each four gene set in the columns B-F. The R² value for each combination is listed only once.

| | Five Gene Fits (R²) | | | | |
|---|---|---|---|---|---|
| | B | C | D | E | F |
| | COMP/ | COMP/ | COMP/ | COMP/ | COMP/ |
| | THS1/ | THS1/ | THS1/ | SIG1/ | SIG1/ |
| Gene | SIG1/ | SIG1/ | IFI44/ | IFI44/ | IFI44/ |
| A | IFI44 | MX1 | MX1 | MX1 | MX1 |
| MX1 | 0.90 | | | | |
| OAS2 | 0.89 | 0.76 | 0.86 | 0.83 | 0.87 |

### Example 6

In the sixth example, 6-gene combinations can be useful for predicting and/or supplementing the mRSS. The correlation between each six gene combination and the mRSS is expressed in the listed R² values of the table. OAS2, the gene listed in column A, is combined with the five gene set listed in column B, and the R² values for the combination is listed where the row for OAS2 listed in column A intersects the column below the five gene set in columns. The R² value for each combination is listed only once.

| Gene | Six Gene Fits (R²) |
|---|---|
| A | B |
| | COMP/THS1/SIG1/IFI44/MX1 |
| OAS2 | 0.90 |

Methods will now be described using these biomarkers that provide objective quantification of the degree of skin disease in patients with systemic sclerosis.

This disclosure provides methods for identifying and using one or more of several genes as biomarkers in order to objectively measure levels of systemic sclerosis. The genes can be isolated from biopsies of the skin of blood mononuclear cells. The identified genes can be used alone or in any combination correlating with and thus predictive of the mRSS in patients with diffuse cutaneous systemic sclerosis. Per one embodiment, a four-gene biomarker (in example 4 above) is used that is highly predictive of the mRSS in patients with diffuse cutaneous systemic sclerosis. Among other useful applications of this method, inclusion of one or more of these biomarkers in clinical assessments and clinical trials of systemic sclerosis skin disease would provide a valuable improved outcome measure. These biomarkers change dynamically in parallel with the mRSS, but do not require any specialized training. They also provide an objective measure of change over time.

According to one embodiment, this disclosure provides a method for identifying genes whose expression correlates with systemic sclerosis, and which can therefore be used as a biomarker for the disease, the method comprises:
(1) Evaluating subjects for systemic sclerosis using the mRSS.
(2) Taking samples from those subjects.
(3) Measuring the levels of expression of genes from the samples.
(4) Normalizing the gene expression levels assayed in the samples.
(5) Calculating changes in the relative expression of each gene.
(6) Determining the statistical significance for differences between systemic sclerosis and control gene expression.
(7) Determining the correlation between genes showing increased expression and the mRSS.
(8) Developing the predictor based on multiple linear regression, evaluating the value of using multiple genes to enhance the correlation and thus predictive power of the biomarker.
(9) Validating the predictor.

Per this embodiment, the subjects used met criteria for diffuse cutaneous systemic sclerosis with proximal skin disease as defined by LeRoy E C, Black C, Fleischmajer R, Jablonska S, Krieg T, et al. (1988) Scleroderma (systemic sclerosis): classification, subsets and pathogenesis. J Rheumatol 15: 202-205. However, other criteria may be used. In this embodiment, the mRSS for each subject is evaluated on the day of the biopsy.

Per this embodiment, 3 mm punch skin biopsies taken from the lesional skin of subjects at a discrete location on the dorsal mid-forearm (either arm). That location is centered along the dorsal width of the forearm, and within two centimeters of the point equidistant between the crook of the wrist and the elbow joint. Samples taken from non-lesional skin were taken from the shoulder or back. Per one example, samples were placed immediately into RNAlater (Qiagen), and stored at −20° C. until preparation of RNA. This RNA preparation occurred within 6 months of the biopsy in the example, although other methods of preservation or time periods may be applied.

In addition to skin biopsies, samples may be taken from other tissues or fluid samples including brochoalveolar lavage fluid, and also including blood samples. [Pendergrass S A, Hayes E, Farina G, Lemaire R, Farber H W, et al. (2010) Limited systemic sclerosis Patients with Pulmonary Arterial Hypertension Show Biomarkers of Inflammation and Vascular Injury. PLoS ONE 5(8): e12106. doi:10.1371/journal.pone.0012106]

One example method for measuring gene expression is measurement of mRNA via RT-PCR. Other known indicators of gene expression may also be employed.

Per one example, RNA is amplified from samples is via an RT-PCR, procedure that comprises: (1) transferring the tissues into 600 μl of RLT buffer (Qiagen, Valencia, Calif.); (2) mincing and disrupting each sample using a Polytron homogenizer; (3) purifying RNAs from RLT buffer supernatants using the RNeasy total RNA kit (Quiagen, Valencia, Calif.); and (4) synthesizing cDNAs from 0.05-0.01 μg of total RNA using Superscript II RNase H-reverse transcriptase and random primers (Invitrogen Life Technologies, Rockville, Md.). Although other methods may be used in this embodiment, RT-PCR is carried out using a Prism 7700 Sequence Detector and primers as recommended by the supplier (Applied BioSystems).

Expression is normalized to 18S rRNA expression (human 18S TaqMan primer set) assayed in the same samples, although other methods may be used.

Changes in the relative expression of each gene may be calculated using the ΔΔCt formula, choosing a healthy donor sample as the control. [Livak K J, Schmittgen T D (2001) Analysis of relative gene expression data using real-time quantitative PCR and the 2(-Delta Delta C(T)) Method. Methods 25: 402-408]. However, other change calculation methods may be employed.

One approach to determine the statistical significance for differences between systemic sclerosis and control mRNA gene expression includes the use the Mann-Whitney test using SPSS (SPSS Inc., Chicago, Ill.). Multiple regression analyses and best-fit modeling may be performed using Minitab 15 Statistical Software (Minitab Inc., State College, Pa.). Further statistical analyses for gene expression correlations with the mRSS and graphing may be carried out using Excel (Microsoft, Seattle, Wash.), although any other methods of calculating may be employed.

Per a four gene biomarker embodiment as described above in example 4, the biomarker skin score was calculated using a four-gene best-fit equation: mRSS=1.49+0.20(COMP)+1.19(THS-1)+0.267(SIG1)+1.59(IFI44), wherein the value for each gene is the fold-change, as determined by RT-PCR and normalized to 18S rRNA.

An approach to validate the predictor involves the use of RT-PCR (or other method for preparation and amplification), to be completed simultaneously on multiple test samples from skin not previously analyzed, as well as on multiple samples that had been amplified previously and that were part of the original predictor which act as internal controls. These internal controls are used to create a regression equation for each gene, comparing the previous and new expression of these controls. Test gene expression levels are normalized using these regression equations, and then used to calculate the biomarker established previously.

The level of gene expression may be determined by measuring the mRNA. The mRNA levels may be measured by a reverse transcriptase polymerase chain reaction method. Oligonucleotide primers suitable for use in the present disclosure include those that can be used in a polymerase chain reaction (PCR) to amplify a nucleotide sequence (e.g. a cDNA sequence) originated from an mRNA encoding a protein of interest. At least one of the PCR primers for amplification of a nucleotide sequence encoding an above-named protein may be sequence-specific for the protein. Primers for use in RT-PCR methods may be intron-spanning where appropriate, in order to avoid amplification of genomic DNA.

Per this embodiment, suitable primers (synthesized by Integrated DNA Technologies) for quantitative real-time polymerase chain reaction (RT-PCR) were designed using Primer Express software (Applied BioSystems). Those exemplary primers were as follows:

For COMP,

forward: 5′ AGC ACC GGC CCC AAG T 3′;
reverse: 5′ GGT TGT GCC AAG ACC ACG TT 3′;

For PAI1,

forward: 5′ AGC TCA TCA GCC ACT GGA AAG 3′;
reverse: 5′ GGA GGA CTT GGG CAG AAC CA 3′;

For THS1,

forward: 5′ CAC AGT TCC TGA TGG AGA ATG C 3′;
reverse: CAT GGA GAC CAG CCA TCG T 3′;

For CTGF,

forward, 5′ TGT GTG ACG AGC CCA AGG A 3′;
reverse: 5′ TCT GGG CCA AAC GTG TCTT C 3′;

For COL4,

forward: 5′GCA AAT GTG ACT GCC ATG GA3′,
reverse: 5′ GAA ACC CAA TGA CAC CTT GTA ACC 3′.

COMP, PAI1 CTGF, COL4 and THS1 mRNA expression values were measured using SYBR Green. To assure the specificity of the COMP, PAI-1 CTGF, COL4 and THS1 primer sets, amplicons generated from PCR reactions were analyzed for specific melting temperatures. For IFI44, MX11, OAS2 and SIG1 TaqMan primers and probes from Applied Biosystems were used.

Biomarker mRNA levels in a sample may also be measured, in one embodiment, by a polynucleotide hybridization method, e.g., Northern blotting. A polynucleotide hybridization method is a method for detecting the presence and/or quantity of a polynucleotide, based on the polynucleotide's ability to form base pairs (under appropriate conditions) with a polynucleotide probe of a known sequence.

In particular embodiments, the amount of biomarker mRNA in a sample is quantitatively determined, following an amplification procedure, e.g. reverse transcriptase polymerase chain reaction (RT-PCR). The level of one of more of the mRNAs is then compared to a standard control. An increase or decrease in the mRNA level for a biomarker is indicative of: the presence or absence of systemic sclerosis, worsening or improvement of systemic sclerosis, or an increased risk of developing systemic sclerosis.

There are numerous methods for extracting mRNA from a biological sample, which may be used alone or in combination. Various commercially available reagents or kits, such as PAXgene Blood RNA Tubes (Qiagen, Valencia, Calif.), Trizol reagent (Invitrogen, Carlsbad, Calif.), Oligotex Direct mRNA Kits, RNeasy Mini Kits (Qiagen, Hilden, Germany), and PolyATtract Series 9600™ (Promega, Madison, Wis.), may be used. The general methods of mRNA preparation (e.g. described by Sambrook and Russell, Molecular Cloning: A Laboratory Manual 3d ed., 2001) can be followed. Combinations of more than one of these methods may also be used.

The amount of mRNA encoding a protein of interest extracted from a sample may be measured, preferably via real-time quantitative PCR, though another amplification-based method may be employed. A DNA copy (cDNA) of the mRNA of interest is first synthesized by reverse transcription in order to facilitate the amplification step. Such synthesis by reverse transcription can be accomplished either in a homogeneous reverse transcription-polymerase chain reaction (RT-PCR), or as a separate step.

Many aspects of PCR are well known in the art and are thus not described in detail herein. PCR may be carried out as an automated process using a thermostable enzyme. Automatic cyclic changes in temperature through ranges allowing for denaturing, primer annealing, and an extension reaction allow for exponential amplification. The machines and reagents required for PCR are commercially available.

PCR amplification of a cDNA derived from the target mRNA may be used in the embodiments of the present disclosure. Other methods of mRNA amplification may be employed, such as ligase chain reaction (LCR), transcription-mediated amplification, and self-sustained sequence replication or nucleic acid sequence-based amplification (NASBA) and branched-DNA signal amplification.

The mRNA of interest can also be detected by using electrophoresis or other methods of size fractionation. These techniques often involve separation on a gel (usually agarose or polyacrylamide) of samples labeled with, for instance, ethidium bromide. If there is a band of the same size as the standard control in the result, that indicates the presence of a target mRNA. The amount of target mRNA obtained can be determined by comparing the band of target mRNA to the control based on the intensity of the band.

The presence of mRNA species, and an indication of the amount of mRNA species in comparison to the standard control, can also be detected using oligonucleotide probes specific to mRNA encoding a biomarker protein.

A normal group of subjects may be selected to establish control levels for each biomarker. A normal subject can be defined as a subject that does not have the condition to be tested for as confirmed, for example, if required, by standard tests appropriate to systemic sclerosis, e.g., mRSS. The control group is preferably large enough for the average amount of mRNA encoding a biomarker calculated from the group to reasonably represent the normal or average amount of that mRNA among those in the general population.

In further embodiments, the method involves detection of the biomarker at the protein level. Accordingly, methods for detecting proteins may include, for example, use of an antibody, capture molecule, receptor, or fragment thereof which selectively binds to the protein. Techniques for production of antibodies to the biomarkers include immunization of an animal and collection of serum (to produce polyclonal antibodies) or spleen cells (to produce hybridomas by fusion with immortalized cell lines leading to monoclonal antibodies). Suitable techniques for detecting protein levels are also known in the art. Those techniques include, but are not limited to, an immunoassay such as an enzyme-linked immunosorbent assay (ELISA), radioimmunoassay (RIA), Western blotting and immunoprecipitation.

Alternatively, the level of the biomarker protein may be determined by mass spectroscopy. Mass spectroscopy allows detection and quantification of a molecule, such as a cytokine protein molecule, by the molecular weight of the molecule. Any suitable ionization method in the field of mass spectroscopy may be employed. Such methods include, without limitation: electron impact chemical ionization (CI), field ionization (FDI), electrospray ionization (ESI), laser desorption ionization (LDI), matrix assisted laser desorption ionization (MALDI) and surface enhanced laser desorption ionization (SELDI). Any suitable mass spectrometry detection method may be employed. Such methods include, without limitation: fourier transform mass spectroscopy (FT-MS), quadrapole mass spectroscopy (QMS), and time-offlight mass spectroscopy (TOF-MS).

Using the methods outlined above, several biomarkers for systemic sclerosis have been identified which correlate well with the mRSS and allow for objective measurement of the disease.

Several of these identified biomarkers are genes regulated by TGFβ, and myofibroblasts, a fibroblast phenotype associated with TGFβ stimulation. Those genes include cartilage oligomeric matrix protein (COMP), connective tissue growth factor (CTGF, also known as CCN2), plasminogen activator inhibitor-1 (PAI1), thrombospondin-1 (THS1) and type IV, alpha 1 collagen (COL4).

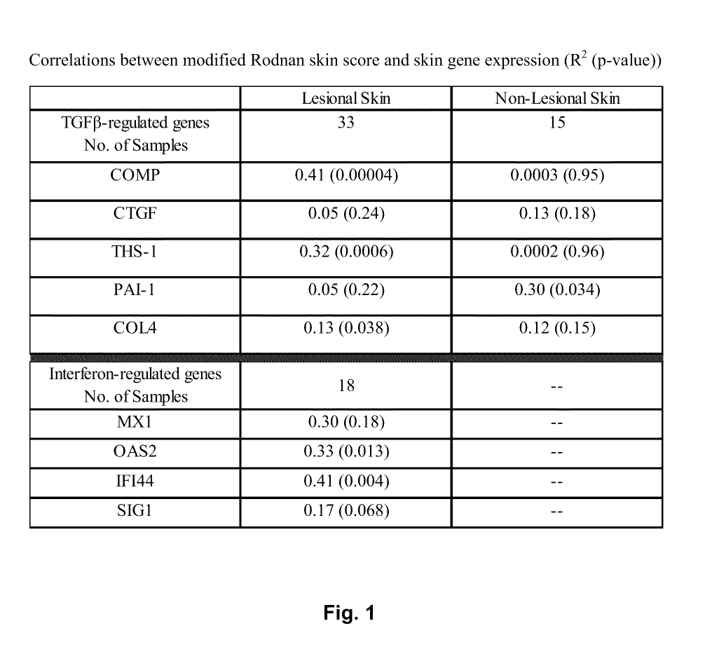

FIG. 1 includes a table which depicts correlations between the mRSS and gene expression in the skin. Various correlation levels between each of these compounds and the mRSS are shown for samples taken from both lesional and non-lesional skin. Of those taken from lesional skin, COMP, TSP-1, and COL4 correlated well with the mRSS (R²=0.41, 0.32, and 0.13 respectively). Of those taken from non-lesional skin, one can see that PAI-1, CTGF, and COL4 each correlated well with the mRSS (R²=0.30, 0.13, and 0.12 respectively). This correlation between expression in non-lesional skin and the mRSS reflects overall skin involvement with systemic sclerosis.

Per one embodiment, mRNA expression of TGFβ-regulated genes, CTGF, THS1, COL4 and PAIL, was measured in lesional (33 biopsies) and non-lesional (15 biopsies) skin from subjects with diffuse cutaneous systemic sclerosis and from healthy controls (5 biopsies). The fold-change was normalized to mRNA expression by one of the healthy controls. The average fold-change compared to this control reference sample of CTGF, THS1, PAI1 and COL4 in lesional diffuse cutaneous systemic sclerosis skin (7.52, 4.08, 9.23 and 4.80, respectively) was compared to the average fold-change in control, healthy skin compared to the control reference sample (2.31, 1.30, 2.37 and 2.43, respectively). The resulting fold increase for CTGF was a 3.25-fold increase (p=0.014); for THS1, 3.14-fold increase (p=0.012); for COL4, 3.89-fold increase (p=0.028); and for PAI1, 1.97-fold increase (p=0.016). PAI1 showed statistically significant increased expression in nonlesional skin compared to healthy control skin (5.08-fold increase, p<0.001).

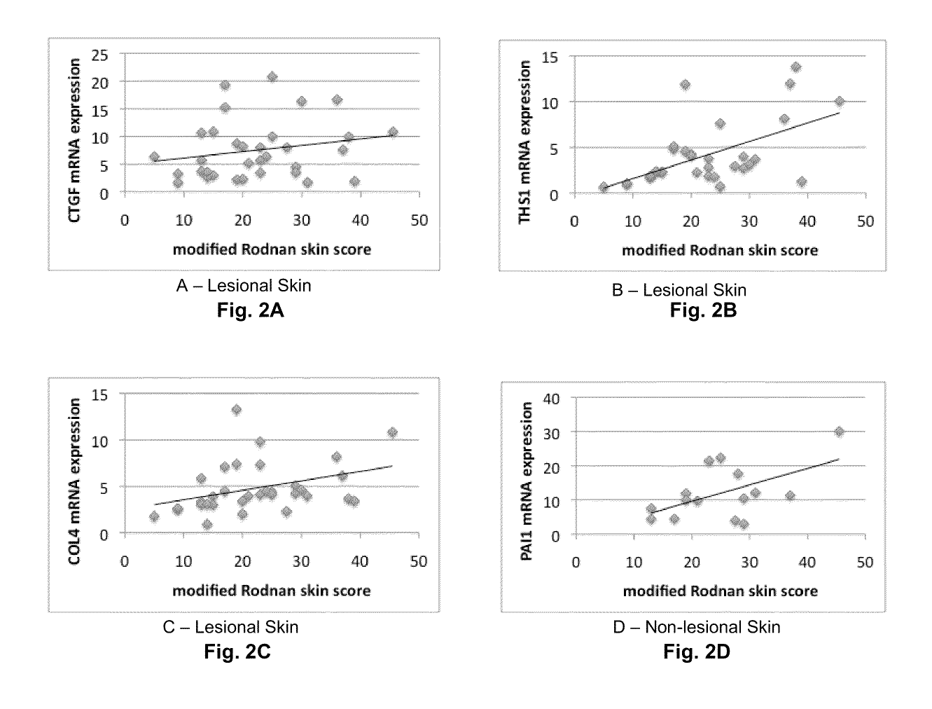

In FIG. 2, which depicts the results of linear regression analysis of the relationship between the mRSS and the expression of TGFβ-regulated genes in the skin of subjects with diffuse cutaneous systemic sclerosis, one can see that there is a correlation between the mRSS and the expression of mRNA for CTGF, THS-1, and COL4 in samples taken from lesional skin, and between the mRSS and the expression of mRNA for PAL1 in samples taken from non-lesional skin.

Several other of the genes identified as biomarkers for systemic sclerosis using the method outlined above are genes regulated by interferon (IFN-regulated genes). Those genes include myxovirus resistance 1 (Mx-1), 2′-5′ oligoadenylate synthetase 2, 69/71 kd (OAS-2), interferon inducible 44 (IFI-44), and sialoadhesin (Siglec-1).

Referring again to FIG. 1, one can see the various correlation levels between each of these compounds and the mRSS for samples taken from lesional skin. MX-1 correlates with an R² value of 0.30; OAS-2 correlates with an R² value of 0.33; IFI-44 correlates with an R² value of 0.41; and Siglec-1 correlates with an R² value of 0.17.

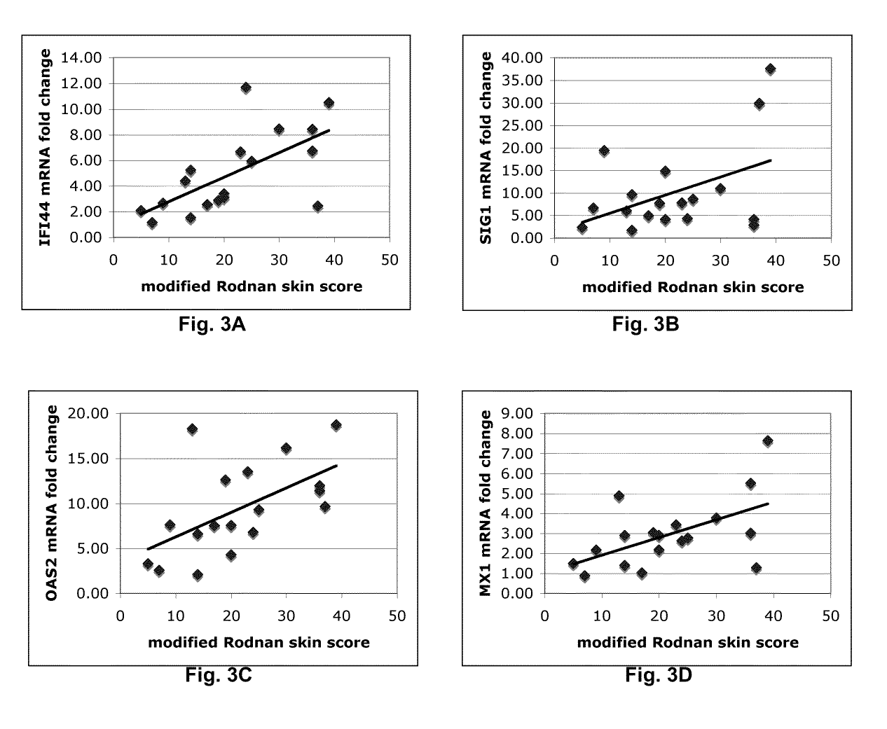

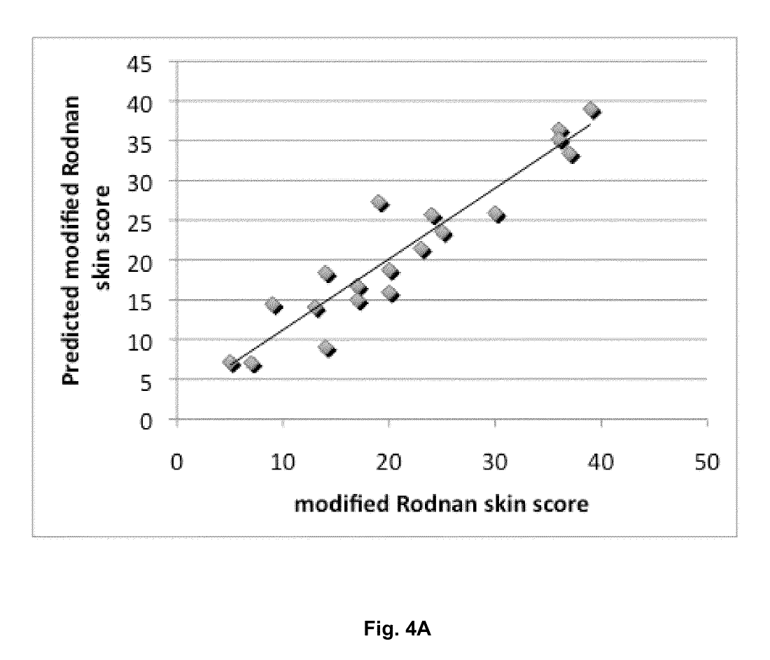

FIGS. 3A-3D depict expression of IFN-regulated genes in relation to corresponding mRSS values taken from the skin of subjects with diffuse cutaneous systemic sclerosis. Linear regression analysis was performed on these values using a simple linear model (graphically depicted in these figures as a straight line), producing the following R2 values an P-values for each one of the analyzed genes: IFI44 (R²=0.41, p=0.004), SIG1 (R²=0.17, p=0.068), OAS2 (R²=0.33 p=0.013) and MX1 (R²=0.30, p=0.018). One can thereby see that the extent of correlation between the mRSS and the expression of mRNA for IFI-44, OAS2, Sig-1, and MX-1.

According to an embodiment, this disclosure provides a method for measuring disease activity in systemic sclerosis, the method comprising: (1) identifying a biomarker which correlates with systemic sclerosis; (2) measuring the level of expression of any such an identified biomarker; and (3) determining whether and to what extent that level of expression is elevated. This disclosure provides for using any of the identified correlating TGFβ regulated genes, including without limitation, COMP, CTGF, PAI1, THS1 and COL4, as such a biomarker. This disclosure also provides for using any of the identified correlating IFN regulated genes, including without limitation, IFI-44, OAS2, Sig-1, and MX-1 as such a biomarker.

Various combinations of multiple markers of TGFβ activity and IFN activity also correlate well with the mRSS. Of these, three- and four-gene regression analyses best-fits that included all or three of the genes COMP, THS1, SIG1 and IFI44 showed high correlations with the mRSS. Including all four of these genes showed a very high correlation with the mRSS, with R²=0.89. The relative weights given to the gene expression in the four-gene best fit equation (set forth above) shows the variability between samples and highlights the requirements for this complimentary set of genes in achieving this accurate predictor of the skin score.

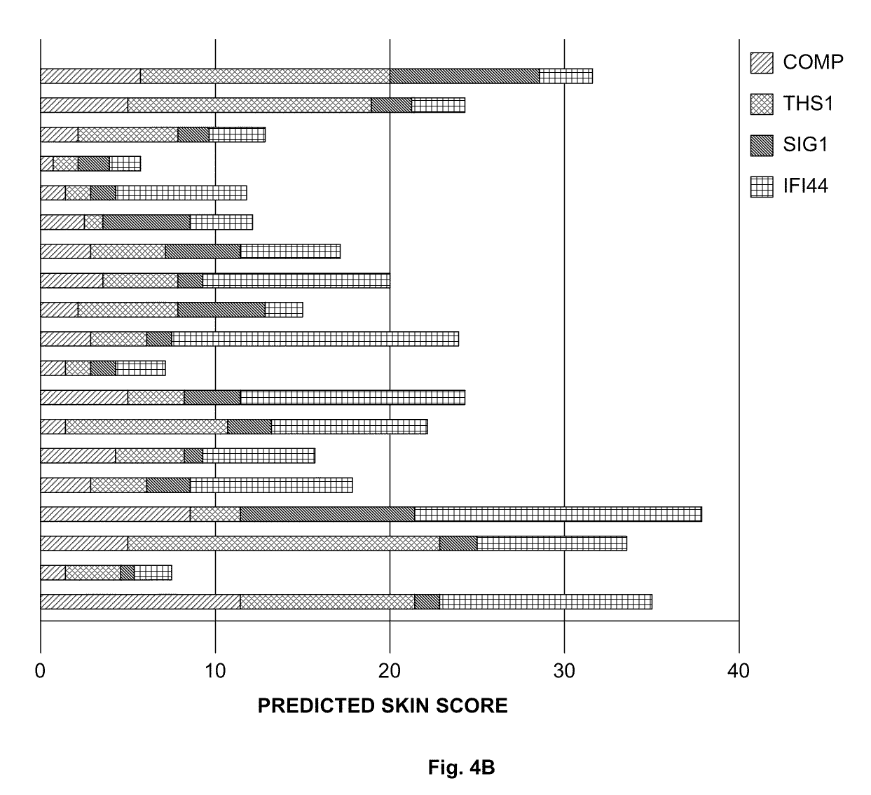

In FIGS. 4A and 4B multiple linear regression of four-gene biomarker with the mRSS in patients with diffuse cutaneous systemic sclerosis is depicted. FIG. 4A depicts best-fit model of skin gene expression of COMP, THS1, IFI44 and SIG1 with the mRSS. FIG. 4B depicts the contribution of expression of each gene, COMP, THS1, IFI44 and 24 SIG1, to the biomarker predicted skin score. Each bar represents the biomarker predicted skin score of one subject.

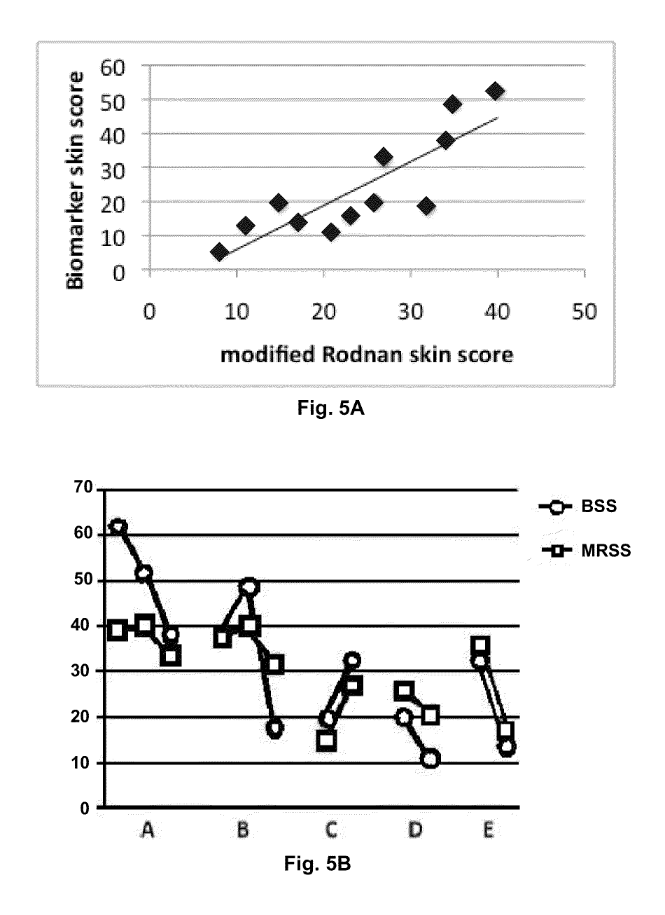

As depicted in FIG. 5, to further validate the utility of this biomarker the regression equation derived from the initial analysis was utilized on a new dataset. Gene expression was measured from 12 additional, lesional skin biopsies, separate from the ones used to generate the predictor. Comparing predicted skin scores with clinical the mRSS confirmed a strong correlation (FIG. 4A, R²=0.73).

Per this embodiment, some of the skin samples in the final series of 12 test and 7 normalization samples tested, included biopsies taken from the same subjects at approximately 6-month intervals. The 4-gene biomarker including COMP, THS1, SIG1, and IFI44, was compared with change in the mRSS to further indicate the utility of the biomarker in longitudinal studies. That four-gene biomarker in all 5 subjects in whom such data was available paralleled the change in skin score. This included both patients showing improvement and progression in skin score (pts. C, D and E of FIG. 5B). In some cases, the biomarker appeared to exaggerate (pt. A) or presage (pt. B) changes detected by the mRSS.

In certain embodiments, this disclosure provides for comparing the biomarker levels in a subject before and after treatment with a therapeutic compound or other therapy. Accordingly, one embodiment provides a method for evaluating whether a treatment is effective in ameliorating, treating or preventing systemic sclerosis, the method comprising: (1) measuring biomarker expression levels in the skin of subjects before treatment, and (2) further measuring biomarker expression levels in the skin of subjects after treatment, wherein a decrease in biomarker expression levels after treatment is indicative of effectiveness.

This disclosure also provides for evaluating a compound formulation. Accordingly, another embodiment provides a method for evaluating whether a formulation is effective in ameliorating, treating or preventing systemic sclerosis, the method comprising: (1) measuring identified biomarker levels in the subjects before treatment with said formulation, and (2) further measuring identified biomarker levels in subjects after treatment with said formulation, wherein a change (e.g., a decrease, where the calculated value increases with disease severity) in identified biomarker levels after treatment is indicative of effectiveness.

This disclosure also provides for evaluating a dose of a drug or a dosage regimen of a drug. Accordingly, another embodiment provides a method for evaluating whether a dosage amount or regime is effective in ameliorating, treating or preventing systemic sclerosis, the method comprising: (1) measuring identified biomarker levels in subjects before treatment with said dosage amount or regime, and (2) further measuring identified biomarker levels in subjects after treatment with said dosage amount or regime, wherein a decrease in identified biomarker levels after treatment is indicative of effectiveness.

Methods of this disclosure are useful in clinical trials, in evaluating the efficacy of a therapeutic regimen, or in monitoring treatment of a subject. Subjects include animals, such as primates, rodents, and birds, (guinea pigs, hamsters, gerbils, rat, mice, rabbits, dogs, cats, horses, pigs, sheep, cows, goats, rhesus monkeys, monkeys, tamarinds, apes, baboons, gorillas, chimpanzees, orangutans, gibbons, chickens, turkeys, ducks, and geese). Zoo, laboratory, and farm animals could be subjects under this disclosure. Mammals, and humans in particular, may be subjects.

In another embodiment, this disclosure provides a method of determining whether a patient is a candidate for therapy for systemic sclerosis, comprising determining identified biomarker levels in the subject, comparing identified biomarker levels in the subject with identified biomarker levels in a normal individual, wherein higher identified biomarker levels in the subject qualifies the subject for therapy.

In yet another embodiment, this disclosure provides a method for predicting the therapeutic outcome of a systemic sclerosis therapy, comprising determining identified biomarker levels in the subject, prior to and after administration of the therapy, wherein a decrease in identified biomarker levels after administration of the therapy is predictive of a potentially successful therapeutic outcome.

Also provided by this disclosure is a method of following the course of therapy for systemic sclerosis comprising the step of monitoring the levels of identified biomarker in the subject at the beginning and during continuation of therapy.

According to another embodiment, this disclosure provides a method for identifying a compound that ameliorates, treats, or prevents an systemic sclerosis, the method comprising: (1) measuring identified biomarker levels in a subject prior to administration of the compound, and (2) further, measuring the levels after administration of the compound, wherein a decrease in the identified biomarker levels after administration of the compound indicates the compound may ameliorate, treat, or prevent systemic sclerosis.

According to another embodiment, this disclosure provides a method for detecting change in systemic sclerosis over time, the method comprising: (1) measuring identified biomarker levels in a subject, and (2) further, measuring the levels at a point later in time, wherein a decrease in the identified biomarker levels after passage of time indicates improvement in systemic sclerosis; and wherein an increase in the identified biomarker levels after passage of time indicates worsening of systemic sclerosis.

In some embodiments kits may be provided. Accordingly, a given kit may be provided which comprises: one or more pairs of oligonucleotide primers for amplifying the sets of biomarkers identified herein as correlating with system sclerosis disease severity and a control sample; and/or one or more reagents for performing RT-PCR. By way of example, the kit may comprise reagents for RNA extraction, reverse transcription (e.g. a reverse transcriptase) or PCR (labeled primers, deoxynucleotides, a thermostable (e.g. Taq) polymerase etc.) as well as appropriate or buffers for performing each of, or any one of, the steps. The kit may further comprise, for example, vials, containers, and/or packaging materials for storing the other items provided. The kit may further comprise instructions for performing one or more method or methods as defined herein.

The methods outlined herein for the identification and measurement of biomarkers for systemic sclerosis may also be modified for other skin diseases, including more common diffuse skin diseases such as psoriasis or atopic dermatitis. Modification would entail correlating the expression of one or more particular genes to an accepted indicator for the level of the particular skin disorder.

For the genes referenced in the embodiments herein, one or more of such genes may be substituted with their variants and mutants, their polynucleotide transcripts, and/or proteins encoded by the genes.

A control level of the mRNA or biomarker may be determined from a single control sample, e.g. from a single normal subject who is known not to suffer from the condition. More preferably, a control level of the mRNA is determined from a mean level in a number of healthy subjects. In certain embodiments, control samples according to the present disclosure contain a known amount of the mRNA encoding a particular protein that closely reflects the average level of such mRNA in a control subject, e.g. a normal healthy individual.

All applications, patents, and references disclosed herein (above and below) are hereby incorporated by reference in their entirety. While we have described a number of embodiments of this disclosure, it is apparent that those embodiments may be altered to provide other embodiments, which utilize the compounds and/or methods of this disclosure. Therefore, it will be appreciated that the scope of this disclosure is to be defined by the appended claims rather than by the specific embodiments, which have been represented by way of example.

What is claimed is:

1. A method for identifying biomarkers for systemic sclerosis, the method comprising:
evaluating subjects for systemic sclerosis using the mRSS;
taking samples from those subjects;
measuring levels of expression of genes from the samples;
normalizing the gene expression levels assayed in the samples;
calculating changes in the relative expression of each gene;
selecting, from among the genes, selected genes for which there are changes in normalized expression levels for subjects with systemic sclerosis in relation to normalized expression levels for the same genes from a control group;
determining the correlation of the selected genes in systemic sclerosis subjects with mRSS values for the systemic sclerosis subjects;
developing a predictor to produce objective severity values based on genes shown to individually correlate with the mRSS values, the objective severity values being indicative a level of severity of systemic sclerosis; and
validating the predictor of the mRSS.

2. A method for measuring systemic sclerosis, the method comprising:
(1) identifying a biomarker which correlates with systemic sclerosis; (2) measuring the level of expression of the identified biomarker; and (2) determining the level of elevated expression of the identified biomarker.

3. The method according to claim 2 wherein the identified biomarker is a TGFβ regulated gene.

4. The method according to claim 2 wherein the identified biomarker is COMP.

5. The method according to claim 2 wherein the identified biomarker is CTGF.

6. The method according to claim 2 wherein the identified biomarker is PAI1.

7. The method according to claim 2 wherein the identified biomarker is THS1.

8. The method according to claim 2 wherein the identified biomarker is COL4.

9. The method according to claim 2 wherein the identified biomarker is an IFN regulated gene.

10. The method according to claim 2 wherein the identified biomarker is IFI-44.

11. The method according to claim 2 wherein the identified biomarker is OAS2.

12. The method according to claim 2 wherein the identified biomarker is Sig-1.

13. The method according to claim 2 wherein the identified biomarker is MX-1.

14. The method according to claim 2 wherein the identified biomarker is a combination of TGFβ regulated genes.

15. The method according to claim 2 wherein the identified biomarker is a combination of IFN regulated genes.

16. The method according to claim 2 wherein the identified biomarker is a combination of TGFβ regulated genes and IFN regulated genes.

17. The method according to claim 2 wherein the identified biomarker is a combination of COMP, THS1, IFI44 and SIG1.

18. A method for evaluating whether a formulation is effective in ameliorating, treating or preventing systemic sclerosis, the method comprising: (1) measuring identified biomarker levels in the subjects before treatment with said formulation, and (2) further measuring identified biomarker levels in subjects after treatment with said formulation, wherein a decrease in identified biomarker levels after treatment is indicative of effectiveness.


SEQUENCE LISTING (10 sequences)

SEQ ID NO: 1 - DNA, 16 bp, Artificial Sequence
  Description of Artificial Sequence Synthetic primer
agcaccggccccaagt

SEQ ID NO: 2 - DNA, 20 bp, Artificial Sequence
  Description of Artificial Sequence Synthetic primer
ggttgtgccaagaccacgtt

SEQ ID NO: 3 - DNA, 21 bp, Artificial Sequence
  Description of Artificial Sequence Synthetic primer
agctcatcagccactggaaag

SEQ ID NO: 4 - DNA, 20 bp, Artificial Sequence
  Description of Artificial Sequence Synthetic primer
ggaggacttgggcagaacca

SEQ ID NO: 5 - DNA, 22 bp, Artificial Sequence
  Description of Artificial Sequence Synthetic primer
cacagttcctgatggagaatgc

SEQ ID NO: 6 - DNA, 19 bp, Artificial Sequence
  Description of Artificial Sequence Synthetic primer
catggagaccagccatcgt

SEQ ID NO: 7 - DNA, 19 bp, Artificial Sequence
  Description of Artificial Sequence Synthetic primer
tgtgtgacgagcccaagga

SEQ ID NO: 8 - DNA, 20 bp, Artificial Sequence
  Description of Artificial Sequence Synthetic primer
tctgggccaaacgtgtcttc

SEQ ID NO: 9 - DNA, 20 bp, Artificial Sequence
  Description of Artificial Sequence Synthetic primer
gcaaatgtgactgccatgga

SEQ ID NO: 10 - DNA, 24 bp, Artificial Sequence
  Description of Artificial Sequence Synthetic primer
gaaacccaatgacaccttgtaacc